SEQUENCE LISTING (6 sequences)

SEQ ID NO: 1 - DNA, 4723 bp, Gallus gallus
  source 1..4723: mol_type = genomic DNA; note = Insertion or deletion of the positions 2993-4547; note = Insertion or deletion of the positions 1210-4443
tctcgtttaggttcccgatgtacagcttgttcatggtggctgccggcggggggcggcgggcgggcgggcggtggcggcgcgccggggctcggagcgcggctgccgggggggctcccaccgtacgcccgcaccgtcggacagcggcctccctctgccaccccacagccggggcgggggctcggtggtccttttctttgaacactccctcccccctcctttttttttttttttttgctttctttttttttgttttggggtgggttttttttttttttttttttttttggtcgttcggcggagttttcctcgcagaggggtgcggcggctgctgctgttgttggggggggggtcctaaaggagggggcggctgcagcgggcgacggctgcgaggagggcggcggcggcgccggctgcggggcccccaaagcggcggagcggcaccggagtgtcaccggacgcctttcggcagccgggcaccgcctctaaataaaaccgacctggaaaatgagtgaaaatgtttgcgggaggaagccacgtggtgatgcgggagggcccggcccccgggggggggggccggggggggagggggcagccgccacttaattatttattttaatttaaatactacgttatttacctccacacattcacacacccgtcttcggagcggggagcgcacccactccgcgttcccctcgcaccccccacctcccccctcgacccttcgggccccgcccgtcccgtcccgtcggaggcactttcccatctgtgcagcctccagcccccaccgcgcccctcccccatcgctcccctcccccacccccttcccccggtgcctgaatgggatttttctgggtgtaaaaaaaaaaaagaaaaagaaaagaaaaaaaaaagagagaaaaaaaaaaaacttcggcatcaaaggggccgggaggtcccctggcgccaggggagcggggctgcaggggaacggcccgacccggcggggtgagcggaggagcccccggggagggctattgtccccggtattgttttaagcccttcatgtgcagccctgcggaagccttcggggaagggggaaggctcctcctggcacaaaagggccccacggtgcagcgggatcgctccccacgggacctggggggggggggacagacgcgccccgtcagcagagagaagcagccccccatccccccgtccctagaaaccctcccttcctctttccctcttcgccttttctggaggctttttatttttaggctcgagatggattgaaaatagcagcgacccccccccccacccccatctcccccccacctccccaaccggcaccacccacccggccgccttcctgctgcctcccccgacggggcgggcgggggaccccggtgcgccgggggctcggcgcgggcggctgccgggcgcggaggggcgggggcggccgtgactccctcccgtgcatgtagaggcacgggccggcccctccgcgcctgcaattaaatgtaacgcgctcctgcctgtcttgcccgccgggccggaggttccgctctccccgtccccccgatcaaaaaaaaataaaaataaaataaaaaataaaataaaataaaataaagaatttccgatttaaataataatagcaataatttttcctccatagaaagaagctttccggttaccctgagcgatatacgggcactgcgcccaccccaccccccccccccgcatcccagccgggggtggcacagtgctgcgatgcggtcgggacggacccccccctcccgcactgctgagccgttcttcgggttctcatcgtccatccggccgctgtggcagcgctgaggggaagagccacgtggagaatttgttagtaaataaatttggtcgtattattcgagaaggagaagttgtgtaaagctttagaaataggcgcacctacccaggggtatttcagtgcgttcagtgctgcggccacgtaggaaactcgtcagaatgaaggattaaaacaaaaacaatctgggaaatttaaagcgaggaaccttcagctctccctcctctgtctgatgcattctgctgtcctggggttttggctttcccaacacagagatgctgctcacagtgggcagtttggtgccatccttccaactgcatcgctctcctcggagcaccaaactgaatccaccagaggggtttgggtggaacttctgtgtatctgtgtgtgtttctgaggcttctgctgcaacctcagctccacattcacaggtgggagggagaaggcaatgaaatttgggaataaatctcctgcagctcccaaagtggctgagaagtggagcgttcaccttcacccacggacacgtcccacaggcacagctagcacaactcatccactgcctgcttcacatccagccctaatctggacccgaatacttctgttgttgctggtttacagagcaccttccctgtgtaacacaaccctttaataatgaaatgaaagaaaccaaccccaaaataacaacaacaacaaaaatcctaacaaagagggaagccttccgtttagacccacaacgaggaaggctatcagtcaacctccttccctcggtggagcgttccagatgtaaggtatttagggaatgcaaacagcagagcccaaatcattgatctgctgcggggcgatagggctgcgctcagccccgggcaggaggaggggcagctgggactgcagggcagctgccccattgagggcaaagctctacaaacccccccgccccccagaagggggaaagggctcaacctcaccaggaacggcagcagtaatccggtgggatacggggagctgagcactatcactcccaacagcagcgtggcctcgttcctcccccagcacttccctgcggttttgagctccctctgaggagcggatttctttgtgcattttacaccacgttccgatgggtccctccacgctctgcagccacgtgaggctgtgtgtgatttcatacccaaaatgtgacccgggctaaaagctggtgggtgtttttcgggggggttgcatggttgatttgttgttgttttgatggcagatttccaccttggaatgaggcagaaaggtccttaatgaatttttaggattactgtacaacaccaaacaattcctgttgtcacacgggagtttaagggaactttcttaaggtaaaataaaaacccaaaaggtgggaactgaatggtgaataatgcacctatgtgtgcaagggatcaatatggaaaggtgcaattcctgcatagtggtgaagtactgttgttgttctgagacagaggaaaattaaaccagaaatgatctctgtggcagagaagtggtgaaatggagtagttcacatcctgatatcttttcaggcagttgccatttgattttgttttaatttgtttggattctttttcggtttggtttggttgcttttgtttttaatgtccggtgatggcactgcatttccagttggacaacacagggaaacacctgaagctctgaacaacgctcatttcccagagctctcatgtataggtaaagttacaatcaaataaagatgggctgaaggatttcactgttgcacttctgtcacggaagggggaaaaaatcactatttgattcatgcagaatctgttacaaacaaacctcagccaccagaaataagccgagcacagagcaaggctgtccctgaaacacaggtgggaactgagcagaatgagaggggtgagggtgcagttggggaatatttcctttctcttacgtgggatcccagttcagaccaccttttgttcatttttttgttaggaggaaaacatttgtgcatggaaaaggaatctcctatgtccaagctgatcccatggattaaagctggagtgcacaaatcagttgagaatcaaacttactgcaggatcaccaattttatttaaaacaaaattccagcctggcattgattattaataatttcctgattctgtgtgcactttggacacagctctgatccccaagggctgcttcctgaagctgaggggcagagcagcatcctgccccggggggaaatgcccctctgccccgggtgggaggggggggctgtaagggtggggggatcccagggggcagtgctgagggtggggatggctcccagtgctggacactgcccgacaaaaagcacaacacaacactgccatcaactcaggctgcagcatcagctcacattctgacaaatccttttgttctgctgctgaaagatggggctccttccaaacaacggcaaaccacaaccaaactcttatttacttctccctccatctgcctgggagctgcttctgtgctcactgcagggcagggtgctctgctcttgcactcctgcagcagcaatctgaattctccactgggactttgcaaacggaccgagtgctgttcagacttcagcaaaacaaagctactttgcatcgatttgtttttaattatttttaatcattttttattctcccccccccccctccacgatggaaatgatttgctttcattttggtgcttcagtccttttctttggctaaaaggtaaatcccaaacacagggagagggaaatgctgactggaaggagccctgaaattgtgcagttcaaagagtccctctaatttttcatttcccccctctttaatgcagctttcaaatatacccccacttcttcccagcagaccttct

SEQ ID NO: 2 - DNA, 3234 bp, Gallus gallus
  source 1..3234: mol_type = genomic DNA; note = Fragment insertion or deletion of the positions 1210-4443
ctcccttcctctttccctcttcgccttttctggaggctttttatttttaggctcgagatggattgaaaatagcagcgacccccccccccacccccatctcccccccacctccccaaccggcaccacccacccggccgccttcctgctgcctcccccgacggggcgggcgggggaccccggtgcgccgggggctcggcgcgggcggctgccgggcgcggaggggcgggggcggccgtgactccctcccgtgcatgtagaggcacgggccggcccctccgcgcctgcaattaaatgtaacgcgctcctgcctgtcttgcccgccgggccggaggttccgctctccccgtccccccgatcaaaaaaaaataaaaataaaataaaaaataaaataaaataaaataaagaatttccgatttaaataataatagcaataatttttcctccatagaaagaagctttccggttaccctgagcgatatacgggcactgcgcccaccccaccccccccccccgcatcccagccgggggtggcacagtgctgcgatgcggtcgggacggacccccccctcccgcactgctgagccgttcttcgggttctcatcgtccatccggccgctgtggcagcgctgaggggaagagccacgtggagaatttgttagtaaataaatttggtcgtattattcgagaaggagaagttgtgtaaagctttagaaataggcgcacctacccaggggtatttcagtgcgttcagtgctgcggccacgtaggaaactcgtcagaatgaaggattaaaacaaaaacaatctgggaaatttaaagcgaggaaccttcagctctccctcctctgtctgatgcattctgctgtcctggggttttggctttcccaacacagagatgctgctcacagtgggcagtttggtgccatccttccaactgcatcgctctcctcggagcaccaaactgaatccaccagaggggtttgggtggaacttctgtgtatctgtgtgtgtttctgaggcttctgctgcaacctcagctccacattcacaggtgggagggagaaggcaatgaaatttgggaataaatctcctgcagctcccaaagtggctgagaagtggagcgttcaccttcacccacggacacgtcccacaggcacagctagcacaactcatccactgcctgcttcacatccagccctaatctggacccgaatacttctgttgttgctggtttacagagcaccttccctgtgtaacacaaccctttaataatgaaatgaaagaaaccaaccccaaaataacaacaacaacaaaaatcctaacaaagagggaagccttccgtttagacccacaacgaggaaggctatcagtcaacctccttccctcggtggagcgttccagatgtaaggtatttagggaatgcaaacagcagagcccaaatcattgatctgctgcggggcgatagggctgcgctcagccccgggcaggaggaggggcagctgggactgcagggcagctgccccattgagggcaaagctctacaaacccccccgccccccagaagggggaaagggctcaacctcaccaggaacggcagcagtaatccggtgggatacggggagctgagcactatcactcccaacagcagcgtggcctcgttcctcccccagcacttccctgcggttttgagctccctctgaggagcggatttctttgtgcattttacaccacgttccgatgggtccctccacgctctgcagccacgtgaggctgtgtgtgatttcatacccaaaatgtgacccgggctaaaagctggtgggtgtttttcgggggggttgcatggttgatttgttgttgttttgatggcagatttccaccttggaatgaggcagaaaggtccttaatgaatttttaggattactgtacaacaccaaacaattcctgttgtcacacgggagtttaagggaactttcttaaggtaaaataaaaacccaaaaggtgggaactgaatggtgaataatgcacctatgtgtgcaagggatcaatatggaaaggtgcaattcctgcatagtggtgaagtactgttgttgttctgagacagaggaaaattaaaccagaaatgatctctgtggcagagaagtggtgaaatggagtagttcacatcctgatatcttttcaggcagttgccatttgattttgttttaatttgtttggattctttttcggtttggtttggttgcttttgtttttaatgtccggtgatggcactgcatttccagttggacaacacagggaaacacctgaagctctgaacaacgctcatttcccagagctctcatgtataggtaaagttacaatcaaataaagatgggctgaaggatttcactgttgcacttctgtcacggaagggggaaaaaatcactatttgattcatgcagaatctgttacaaacaaacctcagccaccagaaataagccgagcacagagcaaggctgtccctgaaacacaggtgggaactgagcagaatgagaggggtgagggtgcagttggggaatatttcctttctcttacgtgggatcccagttcagaccaccttttgttcatttttttgttaggaggaaaacatttgtgcatggaaaaggaatctcctatgtccaagctgatcccatggattaaagctggagtgcacaaatcagttgagaatcaaacttactgcaggatcaccaattttatttaaaacaaaattccagcctggcattgattattaataatttcctgattctgtgtgcactttggacacagctctgatccccaagggctgcttcctgaagctgaggggcagagcagcatcctgccccggggggaaatgcccctctgccccgggtgggaggggggggctgtaagggtggggggatcccagggggcagtgctgagggtggggatggctcccagtgctggacactgcccgacaaaaagcacaacacaacactgccatcaactcaggctgcagcatcagctcacattctgacaaatccttttgttctgctgctgaaagatggggctccttccaaacaacggcaaaccacaaccaaactcttatttacttctccctccatctgcctgggagctgcttctgtgctcactgcagggcagggtgctctgctcttgcactcctgcagcagcaatctgaattctccactgggactttgcaaacggaccgagtgctgttcagact

SEQ ID NO: 3 - DNA, 1555 bp, Gallus gallus
  source 1..1555: mol_type = genomic DNA; note = Fragment insertion or deletion of the positions 2993-4547
atacccaaaatgtgacccgggctaaaagctggtgggtgtttttcgggggggttgcatggttgatttgttgttgttttgatggcagatttccaccttggaatgaggcagaaaggtccttaatgaatttttaggattactgtacaacaccaaacaattcctgttgtcacacgggagtttaagggaactttcttaaggtaaaataaaaacccaaaaggtgggaactgaatggtgaataatgcacctatgtgtgcaagggatcaatatggaaaggtgcaattcctgcatagtggtgaagtactgttgttgttctgagacagaggaaaattaaaccagaaatgatctctgtggcagagaagtggtgaaatggagtagttcacatcctgatatcttttcaggcagttgccatttgattttgttttaatttgtttggattctttttcggtttggtttggttgcttttgtttttaatgtccggtgatggcactgcatttccagttggacaacacagggaaacacctgaagctctgaacaacgctcatttcccagagctctcatgtataggtaaagttacaatcaaataaagatgggctgaaggatttcactgttgcacttctgtcacggaagggggaaaaaatcactatttgattcatgcagaatctgttacaaacaaacctcagccaccagaaataagccgagcacagagcaaggctgtccctgaaacacaggtgggaactgagcagaatgagaggggtgagggtgcagttggggaatatttcctttctcttacgtgggatcccagttcagaccaccttttgttcatttttttgttaggaggaaaacatttgtgcatggaaaaggaatctcctatgtccaagctgatcccatggattaaagctggagtgcacaaatcagttgagaatcaaacttactgcaggatcaccaattttatttaaaacaaaattccagcctggcattgattattaataatttcctgattctgtgtgcactttggacacagctctgatccccaagggctgcttcctgaagctgaggggcagagcagcatcctgccccggggggaaatgcccctctgccccgggtgggaggggggggctgtaagggtggggggatcccagggggcagtgctgagggtggggatggctcccagtgctggacactgcccgacaaaaagcacaacacaacactgccatcaactcaggctgcagcatcagctcacattctgacaaatccttttgttctgctgctgaaagatggggctccttccaaacaacggcaaaccacaaccaaactcttatttacttctccctccatctgcctgggagctgcttctgtgctcactgcagggcagggtgctctgctcttgcactcctgcagcagcaatctgaattctccactgggactttgcaaacggaccgagtgctgttcagacttcagcaaaacaaagctactttgcatcgatttgtttttaattatttttaatcattttttattctcccccccccccctccacgatggaaatgatttgctttcattt

SEQ ID NO: 4 - DNA, 26 bp, synthetic construct
  source 1..26: mol_type = other DNA; note = ASP-F
cccccgtccctagaaaccagcaaaac

SEQ ID NO: 5 - DNA, 21 bp, synthetic construct
  source 1..21: mol_type = other DNA; note = 2K-F
caactcatccactgcctgctt

SEQ ID NO: 6 - DNA, 30 bp, synthetic construct
  source 1..30: mol_type = other DNA; note = ASP-R
gtctgctgggaagaagtgggggtatatttg

(19) United States
(12) Patent Application Publication
(10) Pub. No.: US 2024/0018603 A1
(43) Pub. Date: Jan. 18, 2024

(54) MOLECULAR MARKER OF IGF2BP1 GENE RELATED TO CHICKEN BODY SIZE TRAIT AND USE THEREOF, AND BREEDING METHOD

(71) Applicant: HENAN AGRICULTURAL UNIVERSITY, Henan (CN)

(72) Inventors: Xiangtao KANG, Henan (CN); Wenting LI, Henan (CN); Kejun WANG, Henan (CN); Yadong TIAN, Henan (CN); Guirong SUN, Henan (CN); Ruili HAN, Henan (CN); Ruirui JIANG, Henan (CN); Donghua LI, Henan (CN); Guoxi LI, Henan (CN); Zhuanjian LI, Henan (CN); Xiaojun LIU, Henan (CN); Hong LI, Henan (CN)

(73) Assignee: HENAN AGRICULTURAL UNIVERSITY, Henan (CN)

(21) Appl. No.: 18/479,837

(22) Filed: Oct. 3, 2023

Related U.S. Application Data
(63) Continuation of application No. PCT/CN2021/130299, filed on Nov. 12, 2021.

(30) Foreign Application Priority Data
Apr. 14, 2021 (CN) 202110402071.4

(51) Int. Cl. C12Q 1/6888

(52) U.S. Cl. CPC C12Q 1/6888; C12Q 2600/124; C12Q 1/686

(57) ABSTRACT

The present disclosure relates to a molecular marker of the IGF2BP1 gene related to a chicken body size trait and the use thereof, and a genetic improvement method. In the present disclosure, it is found by means of gene association analysis that a deletion mutation in a promoter region of the IGF2BP1 gene of chicken is significantly associated with the chicken body size trait.

### CROSS-REFERENCE TO RELATED APPLICATION

This application is a continuation of international application of PCT application serial no. PCT/CN2021/130299 filed on Nov. 12, 2021, which claims the priority benefit of Chinese application serial no. 202110402071.4, filed on Apr. 14, 2021. The entirety of each of the above-mentioned patent applications is hereby incorporated by reference herein and made a part of this specification.

### REFERENCE TO A SEQUENCE LISTING

The instant application contains a Sequencing Listing which has been submitted electronically in XML file and is hereby incorporated by reference in its entirety. Said XML copy, created on Aug. 7, 2023, is named 135179_sequencing-listing and is 17,162 bytes in size.

### BACKGROUND

### Field of the Disclosure

The disclosure belongs to biological breeding technical field, and specifically related to the molecular marker of IGF2BP1 gene related to chicken body size trait and use thereof, and breeding method.

### Description of Related Art

In China, there is an abundance of native chicken breeds which have delicious meat, high resistance to stress, etc., but their body size is small and they grow slowly, and therefore they are not able to meet the requirements of modern intensive production. Fast and large white-feathered broilers in foreign countries have fast growth speed and good carcass performance. As a result, China mainly relies on imports of white-feathered broilers as the main source. In view of the above, it is of great significance to cultivate fast and large broiler qualified for obtaining intellectual property rights in China. Body size traits have always been one of the key economic traits in chicken genetic improvement. In conventional breeding, breed is usually retained based on phenotypic traits measurement of individuals, siblings or descendants. However, early selection cannot be carried out through conventional breeding methods, and therefore generation interval is increased and selection response is reduced. In light of the above, developing new molecular markers and speeding up the breeding process with the help of molecular marker-assisted selection has become a new strategy for improving chicken germplasm resources.

With regard to IGF2BP1 gene, the full name thereof is insulin-like growth factor 2 binding protein, and IGF2BP1 gene has the effect of regulating cell proliferation, differentiation, morphogenesis and metabolism. Generally, the mechanism of IGF2BP1 gene is to regulate the localization, stability and translation of mRNA of target genes. In recent years, IGF2BP1 has been confirmed as m⁶A reader, and is likely to widely act on target genes through the regulation mode of m⁶A methylation. IGF2BP1 knockout mice exhibited a reduced body weight and body size. Genome-wide association analysis and QTL mapping studies in chickens and ducks found that the upstream of the IGF2BP1 gene was significantly correlated with carcass and body size traits such as body weight, head weight, breast width, and leg weight. However, the causative mutation of the IGF2BP1 gene that affects chicken body size traits has not yet been elucidated.

### SUMMARY OF THE DISCLOSURE

The purpose of the present disclosure is to provide a molecular marker of IGF2BP1 gene related to chicken body size trait.

The second purpose of the present disclosure is to provide an application of the molecular marker for genetic improvement on chicken body size trait.

The third purpose of the present disclosure is to provide primers and test kits for detecting genotypes of the molecular markers.

The fourth purpose of the present disclosure is to provide a method to improve chicken body size trait.

In order to achieve the above purposes, the present disclosure adopts the following technical solutions:

A molecular marker of IGF2BP1 gene related to a chicken body size trait, a nucleotide sequence thereof is as shown in SEQ ID NO: 1, insertion or deletion is at positions 1210-4443 from the 5′ end or positions 2993-4547 from the 5′ end. A nucleotide sequence at positions 1210-4443 from the 5′ end of the SEQ ID NO: 1 is shown in SEQ ID NO: 2, and a nucleotide sequence at positions 2993-4547 from the 5′ end of the SEQ ID NO: 1 is shown in SEQ ID NO: 3.

An application of the molecular marker in genetic improvement on chicken body size trait.

Preferably, the insertion or deletion of positions 1210-4443 from the 5′ end or 2993-4547 from the 5′ end of the nucleotide sequence shown in SEQ ID NO: 1 is detected, and homozygous chicken individuals with deletion of positions 1210-4443 from the 5′ end of the nucleotide sequence shown in SEQ ID NO: 1 are selected.

A nucleotide sequence of a primer used for detecting genotype of the molecular marker is as shown in SEQ ID NO: 4-6.

A test kit used for detecting the molecular marker genotype includes the primers shown in SEQ ID NO: 4-6.

Preferably, the test kit also includes one or more of dNTPs, PCR reaction buffer, and DNA polymerase.

A method to genetic improvement for the chicken body size trait includes the following steps:
1) extracting a chicken DNA to be tested; designing a primer according to the molecular marker, and utilizing a designed primer to carry out PCR amplification;
2) selecting chicken individuals with homozygous deletion genotype of the positions 1210-4443 from the 5′ end of the nucleotide sequence shown in SEQ ID NO: 1 according to the results of PCR amplification for breeding.

Preferably, in step 1), DNA is extracted by utilizing phenol-chloroform method and is diluted to 60 ng/μL. A nucleotide sequence of the designed primers are shown in SEQ ID NO: 4-6; a band size of the PCR amplification product is detected by agarose gel electrophoresis, and the insertion or deletion of positions 1210-4443 or positions 2993-4547 from the 5′ end of the nucleotide sequence shown in SEQ ID NO: 1 is determined to perform genotype identification. There are six genotypes: WW genotype shows a band at 2344 bp; L2W genotype shows two bands at 2344 bp and 790 bp; L1W genotype shows two bands at 2344 bp and 290 bp; L2L2 genotype shows a band at 790 bp; L1L2 genotype shows two bands at 290 bp and 790 bp; the L1L1 genotype shows a band at 290 bp; chicken individuals with the L1L1 genotype are selected for breeding.

Preferably, in step 1), primers shown in SEQ ID NO: 4 and 6 and primers shown in SEQ ID NO: 5 and 6 are respectively used for a PCR amplification reaction, and a reaction system is as follows:
\textcircled{1}2×Rapid Taq PCR Master Mix 10 μL, ASP-F 0.5 μL, ASP-R 0.5 μL, 1 μL of a chicken DNA template to be tested, ddH₂O 8 μL;
\textcircled{2}2×Rapid Taq PCR Master Mix 10 μL, 2K-F 0.5 μL, ASP-R 0.5 μL, 1 μL of the chicken DNA template to be tested, ddH₂O 8 L.

Preferably, a PCR reaction program in step 1) is carried out as follows: a pre-denaturation is carried out for 5 minutes at 95° C.; a denaturation is carried out for 15 seconds at 95° C., an annealing is carried out for 15 seconds at 60° C., an extension is carried out for 5 seconds at 72° C., and the above process is performed for 30 cycles; an extension is carried out for 5 minutes at 72° C.

The advantageous effect of the present disclosure is as follows:

The disclosure provides a molecular marker of the IGF2BP1 gene associated with chicken body size traits. The inventors discovered for the first time that there were two deletion genotypes at upstream of the IGF2BP1 gene, and PCR and sequencing confirmed that the deletion genotypes are completely consistent with the results of high-throughput sequencing analysis. The present disclosure analyzes the correlation between the deletion mutation in a promoter region of the chicken IGF2BP1 gene and 23 kinds of the chicken body size traits, and it is proved that the allele L1 at this variation site is significantly and positively correlated with high body size traits (23 traits such as claw weight, shin length, sternum length, and double wing weight); and there is a significant positive correlation between the allele W and the chickens low body size traits. The molecular marker of the IGF2BP1 gene provided by the disclosure may be applied to the genetic improvement of the chicken body size traits.

The present disclosure further provides a genetic improvement method for chicken body size trait based on the molecular marker of the IGF2BP1 gene. By extracting chicken DNA to be tested, carrying out PCR amplification of gene fragments corresponding to molecular markers, detecting the band size of PCR amplified products by agarose gel electrophoresis, determining the insertion or deletion of positions 1210-4443 or positions 2993-4547 from the 5′ end of the nucleotide sequence shown in SEQ ID NO: 1 and performing genotype identification, selecting chicken individuals with genotype L1L1 for breeding, thus obtaining chicken germplasm resources with high body size traits, and reaching the goal of genetic improvement of body size traits in chicken flocks. The method may predict the body size traits of chickens in an early stage, and the prediction is fast, inexpensive and effective. In this manner, it is possible to shorten the generation interval and accelerate the genetic progress, and the method has extremely high economic value and broad application prospects.

### BRIEF DESCRIPTION OF THE DRAWINGS

FIG. 1 is a distribution frequency of 1-2 kb deletion and 2-3 kb deletion in a promoter region of chicken IGF2BP1.

In the figure, (A) is 1-2 kb in a promoter region of IGF2BP1; (B) is 2-3 kb in a promoter region of IGF2BP1.

FIG. 2 is a structural variation association analysis of 1-2 kb and 2-3 kb in promoter regions of chicken IGF2BP1.

In the figure, (A) is 1-2 kb in a promoter region of IGF2BP1; (B) is 2-3 kb in a promoter region of IGF2BP1.

FIG. 3 is an allelic structural diagram of a promoter of IGF2BP1.

FIG. 4 shows comparison results of three kinds of allele genome in a promoter region of IGF2BP1.

FIG. 5 is an allele-specific PCR detection result of a promoter region of IGF2BP1.

In the figure, lane M is DM2000 marker, and the marker band size from bottom to top is respectively: 100 bp, 250 bp, 500 bp, 750 bp, 1000 bp, 2000 bp, 3000 bp, and 5000 bp.

FIG. 6 is a PCR amplification condition of a primer of IGF2BP1.

FIG. 7 shows an allele distribution frequency of chromosome structure revealed by PCR and sequencing.

FIG. 8 is correlation analysis between a structural variation genotypes of IGF2BP1 promoter region and body size phenotypes.

### DESCRIPTION OF EMBODIMENTS

The disclosure provides a molecular marker of IGF2BP1 gene associated with chicken body size traits. The inventors discovered for the first time that there are two deletion genotypes at upstream of the IGF2BP1 gene, and PCR and sequencing confirmed that the deletion genotypes are completely consistent with the results of high-throughput sequencing analysis. The molecular marker of IGF2BP1 gene related to chicken body size traits is the insertion or deletion of the nucleotide sequence of the chicken at position chr27: 6082202-6085435 or the insertion or deletion of the nucleotide sequence of the chicken at position chr27: 6083984-6085538.

The application of the molecular marker in genetic improvement of chicken body size trait is as follows. Preferably, the insertion or deletion of the nucleotide sequence of the chicken at position chr27: 6082202-6085435 or the insertion or deletion of the nucleotide sequence of the chicken at position chr27: 6083984-6085538 is determined, the chicken individuals with homozygous deletion genotype at position chr27: 6082202-6085435 are selected for breeding.

Primer used for detecting genotype of the molecular marker, a nucleotide sequence thereof is as shown in SEQ ID NO: 4-6.

A test kit used for detecting genotype of the molecular marker described above includes the primers shown in SEQ ID NO: 4-6.

Preferably, the test kit also includes one or more of dNTPs, PCR reaction buffer, and DNA polymerase.

A genetic improvement method for the chicken body size trait includes the following steps:
1) extracting DNA of chicken to be tested; designing a primer according to the molecular marker, and utilizing the designed primer to carry out a PCR amplification;
2) selecting homozygous chicken individuals with deletion of a nucleotide sequence of the chicken at position chr27: 6082202-6085435 according to results of the PCR amplification for breeding.

Preferably, in step 1), a nucleotide sequence of the designed primer is as shown in SEQ ID NO: 4-6; a band size of PCR-amplified product is detected by utilizing agarose gel electrophoresis; the insertion or deletion of the nucleotide sequence of the chicken at position chr27: 6082202-6085435 or the chicken at position chr27: 6083984-6085538 is determined and genotype identification is carried out; there are six genotypes: WW genotype shows one band of 2344 bp; L2W genotype shows two bands of 2344 bp and 790 bp; L1W genotype shows two bands of 2344 bp and 290 bp; L2L2 genotype shows one band of 790 bp; L1L2 genotype shows two bands of 290 bp and 790 bp; L1L1 genotype shows one band of 290 bp; chicken individuals with genotype L1L1 are selected for breeding.

Further preferably, in step 1), the primers shown in SEQ ID NO: 4 and 6 and the primers shown in SEQ ID NO: 5 and 6 are respectively adopted to carry out a PCR amplification reaction, and a reaction system is as follows.

\textcircled{1}2×Rapid Taq PCR Master Mix 10 μL, ASP-F 0.5 μL, ASP-R 0.5 μL, 1 μL of DNA template of chicken to be tested, ddH₂O 8 μL;

\textcircled{2}2×Rapid Taq PCR Master Mix 10 μL, 2K-F 0.5 μL, ASP-R 0.5 μL, 1 μL of DNA template of chicken to be tested, ddH₂O 8 μL.

Preferably, a PCR reaction process in step 1) is carried out as follows: a pre-denaturation is carried out for 5 minutes at 95° C.; a denaturation is carried out for 15 seconds at 95° C., an annealing is carried out for 15 seconds at 60° C., an extension is carried out for 5 seconds at 72° C., and the above process is performed for 30 cycles; an extension is carried out for 5 minutes at 72° C.

Below in conjunction with specific embodiment the present disclosure is further described, but protection scope of the present disclosure is not limited thereto; all kinds of reagents used in the embodiment and the test example are commercially available goods.

### Embodiment 1 Molecular Marker of IGF2BP1 Gene Related to a Chicken Body Size Trait and an Application Thereof

This embodiment provides a molecular marker of IGF2BP1 gene relevant to chicken body size traits, and the nucleotide sequence thereof is as shown in SEQ ID NO: 1, insertion or deletion of positions 1210-4443 from the 5′ end or positions 2993-4547 from the 5′ end; the nucleotide sequence at positions 1210-4443 from the 5′ end of the SEQ ID NO: 1 is shown in SEQ ID NO: 2, the nucleotide sequence at positions 2993-4547 from the 5′ end of the SEQ ID NO: 1 is shown in SEQ ID NO: 3.

The molecular marker may be applied to genetic improvement on chicken body size trait: detecting the insertion or deletion of positions 1210-4443 from the 5′ end or positions 2993-4547 from the 5′ end of the nucleotide sequence shown in SEQ ID NO: 1; selecting homozygous chicken individuals with deletion of positions 1210-4443 (SEQ ID NO: 2) from the 5′ end of the nucleotide sequence shown in SEQ ID NO: 1, thereby improving the body size traits of the chicken flock.

### Embodiment 2 Detection of Primers of Molecular Marker Genotype

This embodiment provides primers for detecting the molecular marker genotype of IGF2BP1 gene associated with the chicken body size traits, and the nucleotide sequence thereof is as shown in SEQ ID NO: 4-6.

The primer combination of 2k-F and Asp-R is utilized to identify insertion or deletion of the positions 2993-4547 from the 5′ end of the nucleotide sequence shown in SEQ ID NO: 1; the primer combination of Asp-F and Asp-R is utilized to identify the insertion or deletion of the positions 1210-4443 from the 5′ end of the nucleotide sequence shown in SEQ ID NO: 1.

### Embodiment 3 A Test Kit for Detection of a Molecular Marker Genotype

The present embodiment provides the test kit for detecting the molecular marker genotype of IGF2BP1 gene related to chicken body size traits. The test kit includes primers for detection shown in SEQ ID NO: 4-6 provided in Embodiment 2, and also includes one or more of dNTPs, PCR reaction buffer, and DNA polymerase.

### Embodiment 4 Genetic Improvement Method for Chicken Body Size Trait

The present embodiment provides a genetic improvement method for chicken body size trait, and includes the following steps:
1) collecting chicken flock to be tested, taking blood from their wing veins, putting their blood into anticoagulant tube, utilizing phenolic extraction method to extract DNA, and dilute the extracted DNA to 60 ng/μL.
2) carrying out PCR amplification on the target gene containing molecular marker:

The primer combination of 2k-F (SEQ ID NO: 5) and Asp-R (SEQ ID NO: 6) is utilized to identify insertion or deletion of the positions 2993-4547 from the 5′ end of the nucleotide sequence shown in SEQ ID NO: 1; the primer combination of Asp-F (SEQ ID NO: 4) and Asp-R (SEQ ID NO: 6) is utilized to identify the insertion or deletion of positions 1210-4443 from the 5′ end of the nucleotide sequence shown in SEQ ID NO: 1.

PCR reaction system: \textcircled{1}2×Rapid Taq PCR Master Mix 10 μL, ASP-F 0.5 μL, ASP-R 0.5 μL, 1 μL of DNA template, ddH₂O 8 μL; \textcircled{2}2×Rapid Taq PCR Master Mix 10 μL, 2K-F 0.5 μL, ASP-R 0.5 μL, 1 μL of DNA template, ddH₂O 8 μL.

A PCR reaction process: a pre-denaturation is carried out for 5 minutes at 95° C.; a denaturation is carried out for 15 seconds at 95° C., an annealing is carried out for 15 seconds at 60° C., an extension is carried out for 5 seconds at 72° C., and the above process is performed for 30 cycles; an extension is carried out for 5 minutes at 72° C.

3) A detection result of agarose gel electrophoresis: Take 5 μL of each of two PCR reaction products from the same sample, and carry out agarose gel electrophoresis.

The W allele band size is 2344 bp, the L1 allele band size is 290 bp, and the L2 allele band size is 790 bp. There are six genotypes: WW genotype shows one band of 2344 bp; L2W genotype shows two bands of 2344 bp and 790 bp; L1W genotype shows two bands of 2344 bp and 290 bp; L2L2 genotype shows one band of 790 bp; L1L2 genotype shows two bands of 290 bp and 790 bp; L1L1 genotype shows one band of 290 bp.

4) Individuals with genotype L1L1 are selected for breeding according to the genotyping result in combination with genetic process, thereby improving the body size traits of the chicken flock.

### Test Example 1

The present disclosure analyzed and verified the correlation between the deletion mutation in a promoter region of the chicken IGF2BP1 gene and the chicken body size traits.

1. Correlation analysis of structural variations and deletion variations in promoter region of chicken IGF2BP1 discovered through high-throughput sequencing

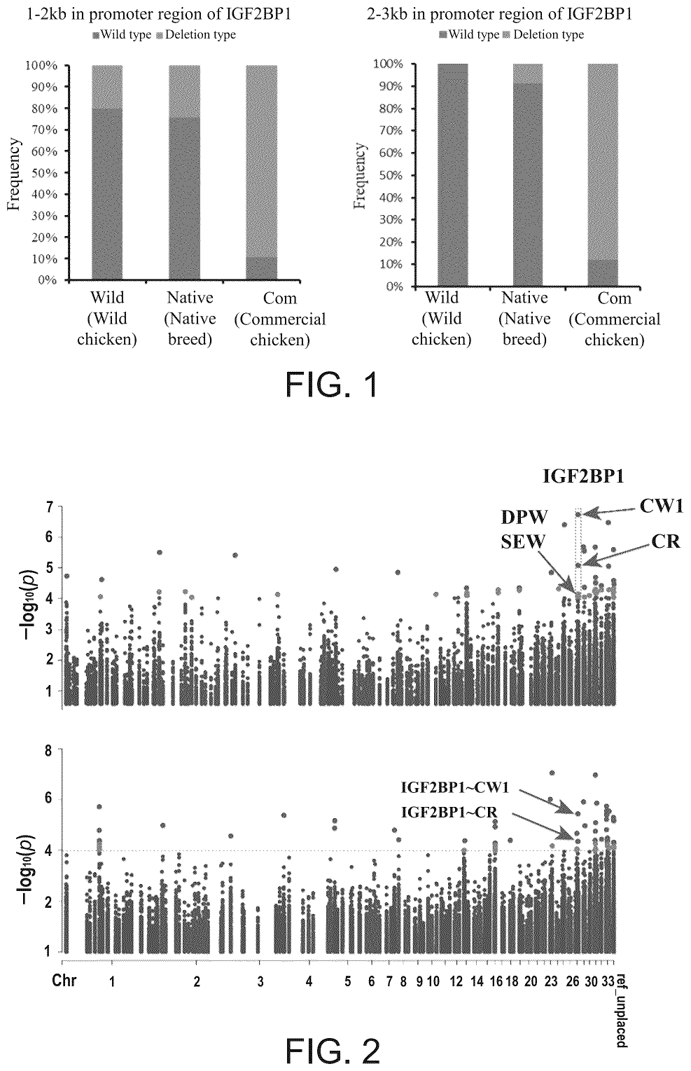

By using whole genome retesting data and by comparatively analyzing genome depth and coverage, identification of structural variations in genomic regulatory regions was carried out by using a 1 kb window size, and it was found that there were deletion variations (mutant type and wild type) in the promoter region of IGF2BP1 gene and there was a significant frequency difference between deletion type (that is mutant type) and wild type in local and commercial chicken breeds (see FIG. 1). Based on the analysis of F2 population association, it was found that the structural variation is significantly associated with chicken body size traits (see FIG. 2).

2. PCR detection was carried out to identify three kinds of structural variation alleles in the promoter region of IGF2BP1.

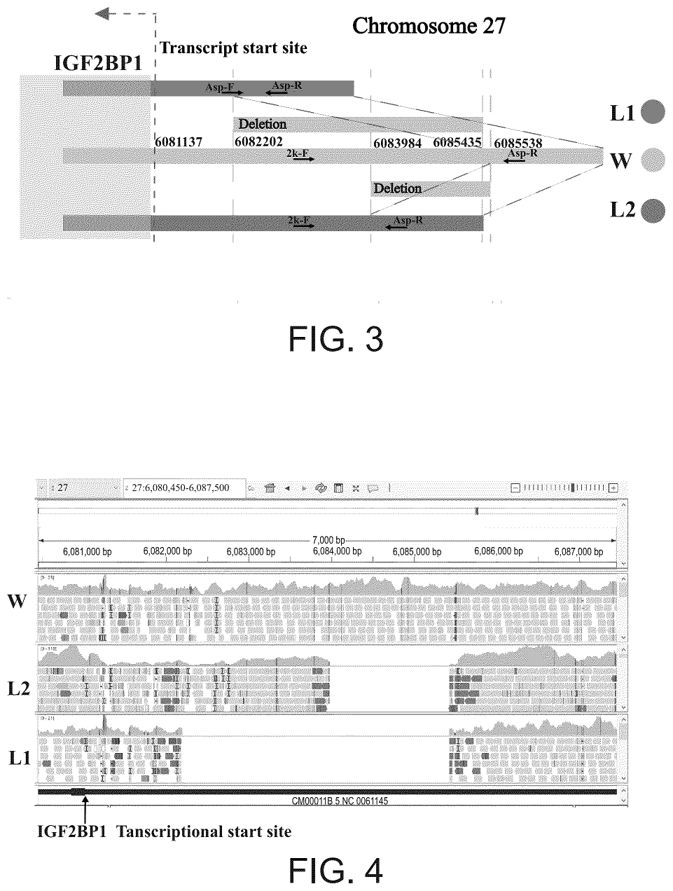

The promoter regions of IGF2BP1 gene of different chicken breeds were detected, and this structural variation had been genotyped by PCR and sequencing, 3 alleles were found altogether, and the mutant type (L1 type and L2 type) was: the L1 type corresponded to a deletion at position chr27: 6082202-6085435 in the chicken reference genome GRCg6a, the length of the deletion was 3234 bp (that is, the positions 1210-4443 from the 5′ end of the sequence shown in SEQ ID NO: 1 is deleted), the L2 type corresponded to a deletion at position chr27:6083984-6085538 in the chicken reference genome GRCg6a, the length of the deletion was 1555 bp (that is, the positions 2993-4547 from the 5′ end of the sequence shown in SEQ ID NO: 1 was deleted); there was no deletion at this position in the wild type of chicken (see FIG. 3 and FIG. 4).

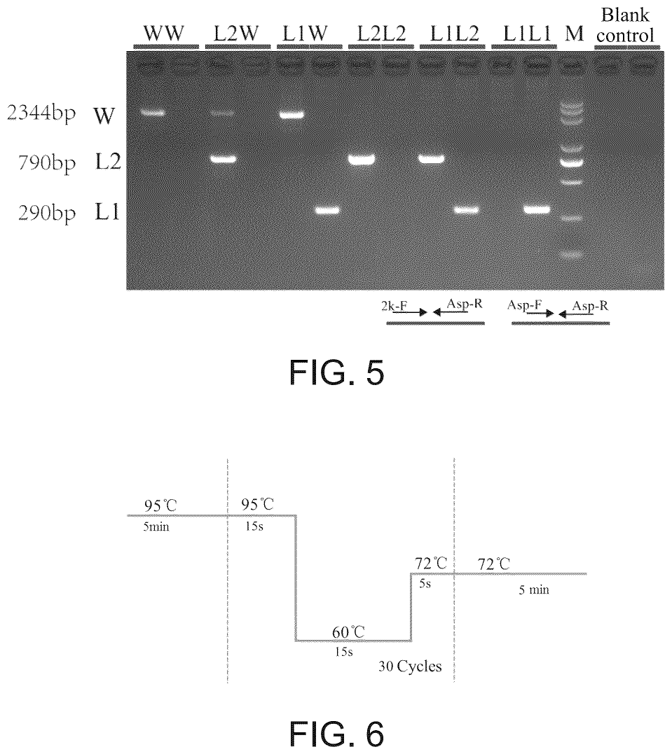

Allele-specific PCR technology was utilized to identify three kinds of allele distributions, and a primer sequence and a condition are as shown in Table 1 and FIG. 5. The W and L2 alleles were identified with the primer combination of 2k-F and Asp-R based on the designed physical location and amplification combination of the primers (see FIG. 3); the primer combination of Asp-F and Asp-R was utilized to identify the L1 allele.

TABLE 1
Three allele-specific primers for promoter
of IGF2BP1

| Primer ID | Primer sequence (5′--3′) |
|---|---|
| 2K-F | CAACTCATCCACTGCCTGCTT |
| ASP-F | CCCCCGTCCCTAGAAACCAGCAAAAC |
| ASP-R | GTCTGCTGGGAAGAAGTGGGGGTATATTTG |

The PCR sample was DNA, and concentration was diluted to 60 ng/μL. Primer concentrations were all 10 μM.

PCR system: 2K-F&ASP-R and ASP-F&ASP-R were utilized respectively to PCR amplification:
\textcircled{1}2×Rapid Taq PCR Master Mix 10 μL, ASP-F 0.5 μL, ASP-R 0.5 μL, 1 μL of DNA template, ddH₂O 8 μL;
\textcircled{2}2×Rapid Taq PCR Master Mix 10 μL, 2K-F 0.5 μL, ASP-R 0.5 μL, 1 μL of DNA template, ddH₂O 8 μL;
2×Rapid Taq PCR Master Mix was purchased from Nanjing Nuoweizan, and primers were synthesized from Sangon Biotech (Shanghai) Co., Ltd.

A PCR reaction process: a pre-denaturation was carried out for 5 minutes at 95° C.; a denaturation was carried out for 15 seconds at 95° C., an annealing was carried out for 15 seconds at 60° C., an extension was carried out for 5 seconds at 72° C., and the above process was performed for 30 cycles; an extension was carried out for 5 minutes at 72° C. (see FIG. 6).

Detection result of agarose gel electrophoresis: Take 5 μL of each of two PCR reaction products from the same sample, and carry out agarose gel electrophoresis. The band size of the W allele was 2344 bp, the band size of the L1 allele was 290 bp, and the band size of the L2 allele was 790 bp. WW genotype showed one band of 2344 bp; L2W genotype showed two bands of 2344 bp and 790 bp; L1W genotype showed two bands of 2344 bp and 290 bp; L2L2 genotype showed one band of 790 bp; L1L2 genotype showed two bands of 290 bp and 790 bp; L1L1 genotype showed one band of 290 bp (see FIG. 5).

3. Allele PCR detection verification results in a promoter region of IGF2BP1

The above allele-specific PCR was utilized to carry out the identification of molecular marker genotype to 734 individuals of Gushi×Anak F2 generation resource group. After identification, there was one-to-one correspondence with the body size traits of Gushi×Anak F2 generation resource group, that is, one individual corresponded to one genotype data and one set of phenotype data. The data was entered into SPSS software, the general linear model was selected, and genotype data was added as fixed effects. The results of the correlation analysis of the general linear model of the single-point variation of the Gushi×Anak F2 generation resource group were obtained by analysis. If the p value of the analysis result was less than 0.05, it would be considered that the variation site was significantly correlated with the trait.

The result showed that genotype L1L1 was all significantly positively correlated with 23 body size traits in this variable site. The 23 body size traits are claw weight, claw rate, tibia length at 12 weeks old, sternum length at 8 weeks old, sternum length at 12 weeks old, double wing weight, eviscerated weight, half eviscerated weight, head weight, carcass weight, leg weight, leg muscle weight, pelvic width at 12 weeks old, shin girth at 8 weeks old, shin girth at 12 weeks old, body oblique length at 8 weeks old, body oblique length at 12 weeks old, gizzard weight, body weight at 6 weeks old, body weight at 10 weeks old, body weight at 12 weeks old, weight after hair removal, growth rate at 0-4 weeks old.

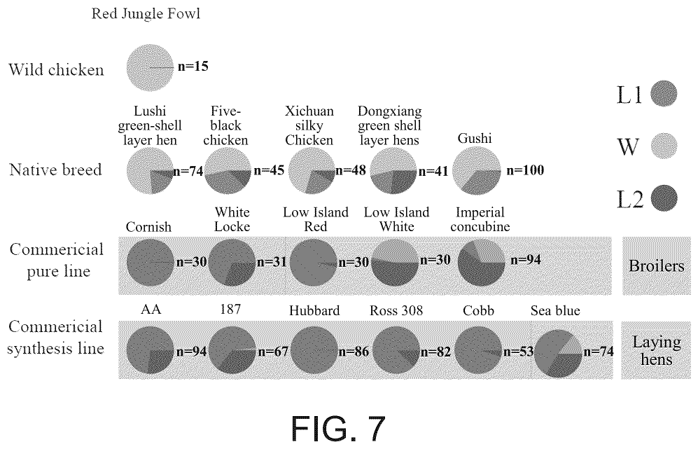

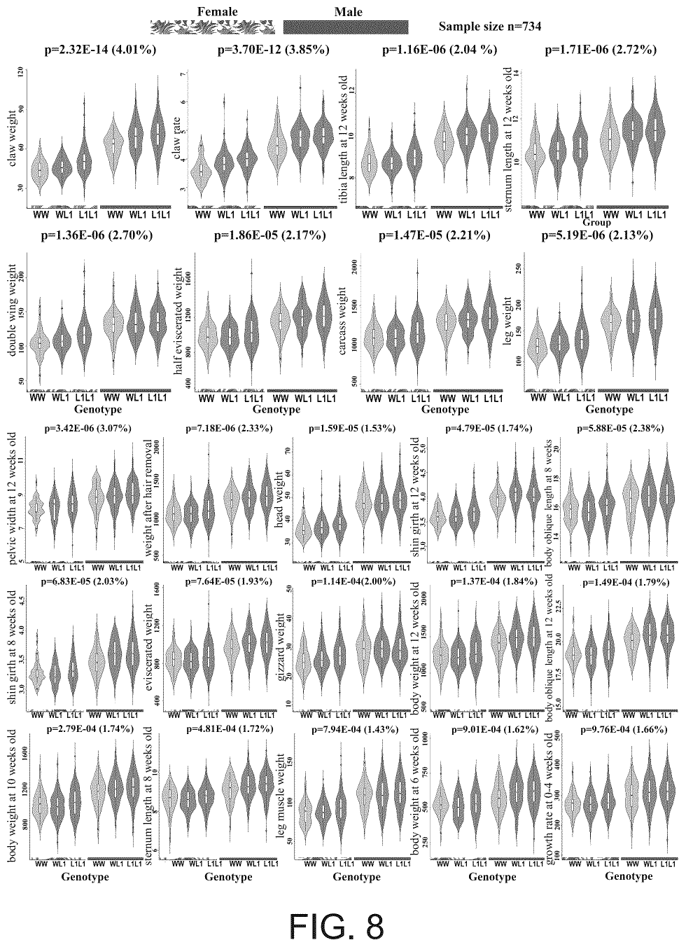

Wild-type allele W mainly existed in red jungle fowl, native chicken breed, and L1 and L2 allele mainly existed in meat commercial pure line and synthetic line, and distribution frequency was as shown in FIG. 7. Based on the general linear model association analysis of the single-point variation of the Gushi×Anak F2 generation resource group, it was found that there was no allele L2 type in this association group. The allele L1 was significantly and positively correlated with chicken height traits (23 traits such as claw weight, shin length, sternum length, and double wing weight). The allele W was significantly positively correlated with the low body size traits of the chicken, and the genotype L1L1 was significantly positively correlated with the high body size traits (23 traits such as claw weight, shin length, sternum length, and double wing weight). The correlation analysis results were shown in FIG. 8.

What is claimed is:

1. A molecular marker of a IGF2BP1 gene related to chicken body size traits, comprising an insertion or deletion of a nucleotide sequence shown in SEQ ID NO: 2, or an insertion or deletion of a nucleotide sequence shown in SEQ ID NO: 3.

2. An application of the molecular marker as claimed in claim 1 in genetic improvement on the chicken body size traits.

3. The application as claimed in claim 2, comprising determining an insertion or deletion of the nucleotide sequence shown in SEQ ID NO: 2 or SEQ ID NO: 3, and selecting homozygous chicken individuals with the deletion of the nucleotide sequence shown in SEQ ID NO: 2 for breeding.

4. A genetic improvement method for chicken body size traits, comprising the following steps:
1) extracting chicken DNA to be tested; designing a primer according to the molecular marker as claimed in claim 1, and utilizing the designed primer to carry out PCR amplification; and
2) selecting homozygous chicken individuals with the deletion of the nucleotide sequence shown in SEQ ID NO: 2 according to PCR amplification results for breeding.

5. The genetic improvement method for the chicken body size traits as claimed in claim 4, wherein in step 1), a nucleotide sequence of the designed primer is shown in SEQ ID NO: 4-6; a band size of a PCR amplification product is detected by agarose gel electrophoresis, and the insertion or the deletion of the nucleotide sequence shown in SEQ ID NO: 2 or SEQ ID NO: 3 is determined to perform genotype identification; there are six genotypes: WW genotype shows a band of 2344 bp; L2W genotype shows two bands of 2344 bp and 790 bp; L1W genotype shows two bands of 2344 bp and 290 bp; L2L2 genotype shows a band of 790 bp; L1L2 genotype shows two bands of 290 bp and 790 bp; and L1L1 genotype shows a band of 290 bp; and chicken individuals with the L1L1 genotype are selected for breeding.

6. The genetic improvement method for the chicken body size traits as claimed in claim 4, wherein in step 1), primers shown in SEQ ID NO: 4 and 6 and primers shown in SEQ ID NO: 5 and 6 are respectively used for a PCR amplification reaction, and a reaction system is as follows:
\textcircled{1}D2×Rapid Taq PCR Master Mix 10 μL, ASP-F 0.5 μL, ASP-R 0.5 μL, 1 μL of a chicken DNA template to be tested, ddH₂O 8 μL;
\textcircled{2}2×Rapid Taq PCR Master Mix 10 μL, 2K-F 0.5 μL, ASP-R 0.5 μL, 1 μL of the chicken DNA template to be tested, ddH₂O 8 μL.

7. The genetic improvement method for the chicken body size traits as claimed in claim 6, wherein a PCR reaction process in step 1) is carried out as follows: a pre-denaturation is carried out for 5 minutes at 95° C.; a denaturation is carried out for 15 seconds at 95° C., an annealing is carried out for 15 seconds at 60° C., an extension is carried out for 5 seconds at 72° C., and the above process is performed for 30 cycles; an extension is carried out for 5 minutes at 72° C.
